(19) United States
(12) Patent Application Publication
(10) Pub. No.: US 2023/0149559 A1
(43) Pub. Date: May 18, 2023

(54) SUPRAMOLECULAR CELL-BASED CARRIER, DRUG LOADING SYSTEM AND ITS PREPARATION METHOD

(71) Applicant: University of Macau, Macau (CN)

(72) Inventors: Ruibing WANG, Macau (CN); Cheng GAO, Macau (CN); Qian CHENG, Macau (CN); Mingyuen LEE, Macau (CN)

(73) Assignee: University of Macau, Macau (CN)

(21) Appl. No.: 18/154,229

(22) Filed: Jan. 13, 2023

Related U.S. Application Data
(63) Continuation of application No. PCT/CN2021/105845, filed on Jul. 12, 2021.

(30) Foreign Application Priority Data
Jul. 14, 2020 (CN) 202010677793.6

(51) Int. Cl. A61K 47/69; A61K 47/60

(52) U.S. Cl. CPC A61K 47/6901; A61K 47/6949; A61K 47/6951; A61K 47/60; A61K 47/6911

(57) ABSTRACT

Disclosed is a method for preparing supramolecular cell-based carrier, which relates to the technical fields of supramolecular chemistry, supramolecular materials and cell preparations. Host-guest interactions mediated supramolecular cell-based carriers can achieve targeted delivery based on cell physiological functions, and have high biocompatibility, physiological barrier permeability, and targeting delivery efficiency. It does not require covalent bond modification on the cell surface, and has no effect on the physiological functions of transporting cells. The preparation method of supramolecular cell-based carrier provided by the present application has the advantages of simple and fast construction process, mild conditions and universal applicability, and the method has bio-orthogonality. In addition, a drug loading system is also provided, which can realize drug loading for targeted therapy.

### CROSS-REFERENCE TO RELATED APPLICATIONS

This present application is a continuation of International Application No. PCT/CN2021/105845 filed on Jul. 12, 2021, which claims the priority to Chinese Patent Application No. 202010677793.6 and entitled “SUPRAMOLECULAR CELL-BASED CARRIER, DRUG LOADING SYSTEM AND ITS PREPARATION METHOD” submitted to the NATIONAL INTELLECTUAL PROPERTY ADMINISTRATION, PRC on Jul. 14, 2020, all of which are incorporated by reference in this application.

### TECHNICAL FIELD

The application relates to the technical fields of supramolecular chemistry, supramolecular material and cell-based formulation, in particular, to a supramolecular cell-based carrier, drug loading system and its preparation method.

### BACKGROUND

Inflammation is closely related to various diseases in humans, including cancer and neurological disease. However, traditional drug formulations and synthetic targeting formulations have no obvious therapeutic effect on these major diseases, which may be attributed to the quick clearance by mononuclear phagocytic system during blood circulation, the weak penetration ability through physiological barrier, and the low targeting delivery efficiency to lesion tissue. These challenges limit the final drug concentration in lesion tissue and affect its therapeutic efficacy. Therefore, it is important to find new delivery vehicles and targeting pathways to meet the requirement of these inflammation-related severe diseases, and develop a new generation of pharmaceutic formulations with high biocompatibility, physiological barrier permeability, and targeting delivery efficiency, which are also the major problems desired to be addressed in the research and clinical practice.

Thus, this application is proposed.

### SUMMARY

The purpose of this application is to provide a supramolecular cell-based carrier, drug loading system and its preparation method to solve the above-mentioned technical problems.

This application is implemented like this:

A supramolecular cell carrier includes a first part and a second part connected to each other through host-guest interaction. The first part is a first cell modified by macrocyclic host molecules or guest molecule, and the second part is a nanoparticle modified by guest molecules or macrocyclic host molecules, or a second cell modified by guest molecules or macrocyclic host molecules. The macrocyclic host molecules or guest molecule in the first part are embedded in the cell membrane of first cell by coupling the membrane-embedding material.

Cells are the basic units that constitute the structure of organisms and carry out biological functions. Using cells as drug delivery vehicles has many natural advantages. However, there are few studies to use cells as drug carriers, and there are also unavoidable defects in the reported methods of constructing cell-based carriers. One of them directly utilizes direct drug internalization to realize its loading progress. However, the phagocytized drug carriers may be degraded in the intracellular environment and cause cytotoxicity, thereby affecting physiological function mediated cell-based delivery. Another method is to conjugate drug carriers on the cell surface through a covalent bond or specific ligand-receptor binding, involving complex and multi-step chemical reactions on cell membrane leading to the cell damage. While the ligand-receptor interaction is limited to specific cells expressing relevant receptors, and its application is relatively limited.

The inventors creatively provided a method of embedding the membrane inserting materials into the cell membrane of first cell, which avoids the decrease of cell activity caused by the covalent binding between the surface and drug carrier, and is also not limited to specific cells expressing relevant receptors. The provided first cells in present application can be adaptively adjusted according to drug loading requirements, and have a wide applications. The other end of the membrane-embedded material is coupled with a macrocyclic host molecule or guest molecule, and the macrocyclic host molecule or guest molecule can be connected with a guest molecule through host-guest interaction to form a supramolecular cell-based carrier.

The membrane-embedded material is similar to the structural components of cell membrane, and is self-assembled with the phospholipid layer on the membrane surface through hydrophobic forces.

Host-guest chemistry is a new research direction that has emerged in recent years. The host molecules bind with guest molecules through non-covalent bonds. Most of the research is using the host-guest chemistry of β-cyclodextrin and adamantane. The guest molecule adamantane in water will automatically combine with the hydrophobic cavity of the host molecule cyclodextrin due to its hydrophobicity, forming a relatively stable host-guest interaction product.

Supramolecular cell-based carriers are constructed through the coupled membrane-embedded material and macrocyclic host molecule or guest molecule embedded in the first cell construction, and subsequent host-guest interaction. It can achieve targeted delivery effect based on cell physiological function. The supramolecular cell-based carriers overcome the defect in the prior art that cells load the drug carrier through the endocytosis of cells, and the supramolecular cell carriers do not cause cytotoxicity and have bio-orthogonality.

In a preferred embodiment of this application, the above-mentioned macrocyclic main molecule is cyclodextrin (CD), cucurbituril (CB), calixarene, pillararene (PA) or crown ether.

The crown ether may be any one of bicyclic crown ether, tricyclic crown ether, polycyclic crown ether and heterocrown ether.

The above-mentioned macrocyclic host molecule has a relatively high binding constant with many guest molecules, which helps to improve the stability of host-guest complex in vivo.

The macrocyclic host molecule or guest molecule is located on the outer surface layer of cell membrane, and the phospholipid which is linked to the macrocyclic molecule or guest molecule fuses with the cell membrane phospholipid bilayer to be embedded in the cell membrane.

In a preferred embodiment of this application, the above-mentioned first cells are selected from any one of macrophages, neutrophils, red blood cells, stem cells, lymphocytes, dendritic cells, platelets and fat cells.

Different cell lines have different physiological functions, such as the inflammatory tropism of immune cells and the homing effect of stem cells, etc. The different physiological functions of these cells also endow the corresponding cells with a strong intrinsic targeting driving force. The appropriate type of cells could be selected as the targeted delivery carrier according to the disease pathological characteristics. For the supramolecular cell-based carrier provided in this application, the corresponding first cell can be selected as required.

Macrophages can be M1 or M2 macrophages. Lymphocytes can be at least one of T cells, B cells, and NK cells.

Fat cells can be white fat cells or brown fat cells.

In a preferred embodiment of this application, the molar ratio of the macrocyclic host molecule to the guest molecule is 1-10:1-10; preferably 1:1;

Preferably, the guest molecule is adamantane or ferrocene.

The macrocyclic host molecule and the guest molecule can simply and quickly realize the preparation of supramolecular carriers under the molar ratio mentioned above.

The guest molecule needs to match the host molecule, and in other embodiments, it can also be replaced as needed.

In a preferred embodiment of this application, the above-mentioned nanoparticles are at least one of liposomes, micelles, nanogels, inorganic nanoparticles and nanocapsules.

Preferably, the second cells are hepatocytes, stem cells, lymphocytes, dendritic cells, platelets and adipocytes or red blood cells.

In other embodiments, all cells whose surfaces could be embedded with “DSPE-PEG-guest molecules” or “DSPE-PEG-macrocyclic host molecule” can be used as a second cell.

Liposomes facilitate the transmembrane transport of supramolecular cell-based carriers and achieve targeted drug delivery through similar polarity. The second cell is liver cells or red blood cells, which is beneficial to improve the targeted therapy ability of supramolecular cell-based carriers for major diseases.

In a preferred embodiment of the application, the above-mentioned embedded membrane material is PEG-DMPE, PEG-DPPE, PEG-DSPE or PEG-CHOL.

PEG-DMPE is PEG-1,2-dimyristoyl-sn-glycero-3-phospho-ethanolamine. PEG-DPPE is PEG-1,2-Dipalmitoyl-sn-glycero-3-phosphoethanolamine. PEG-DSPE is PEG-1,2-distearoyl-sn-glycero-3-phosphoethanolamine, and PEG-CHOL is PEG-cholesterol.

In one embodiment, DSPE-PEG-ADA, cholesterol, and lecithin can be used to prepare liposomes with a surface enriched in ADA (adamantane).

In another embodiment, the membrane-embedding effect of D SPE-PEG-ADA can be used to construct hepatocytes with surface-modified guest molecule adamantane.

A method for preparing a supramolecular cell carrier, which includes: co-incubating a macrocyclic host molecule or guest molecule coupled with a membrane-embedded material with a first cell to obtain the first part, and then adding nanoparticles modified with guest molecules or macrocyclic host molecules, or second cells modified with guest molecules or macrocyclic host molecules. The modified nanoparticles or second cells are mixed with the first part to get the supramolecular cell-based carrier.

The preparation method of supramolecular cell-based carrier provided by this application has the advantages of a simple and rapid preparation process, mild conditions, universal applicability, and the method has bio-orthogonality.

In a preferred embodiment of this application, the above preparation method further includes first coupling the macrocyclic host molecule to the membrane-embedding material, and then embedding the macrocyclic host molecule coupled to the membrane-embedding material into the cell membrane of first cell;

Preferably, the macrocyclic host molecule is covalently linked to PEG in the membrane-embedding material; preferably, the co-incubation time of the macrocyclic host molecule coupled with the membrane-embedding material and the first cell is more than 30 minutes; The concentration of the macrocyclic host molecule coupled with membrane embedding material is 1 μM-1 mM.

In other embodiments, macrocyclic host molecules coupled with membrane-embedding materials or nanoparticles modified with guest molecules can also be purchased directly.

In a preferred embodiment of this application, the above-mentioned nanoparticles modified with guest molecules or the second cells modified with guest molecules are mixed and incubated with the first part for ≥10 seconds.

In a drug loading system, the carrier system includes supramolecular cell-based carriers and drugs, and the drugs are loaded in nanoparticles or in second cells; preferably, the nanoparticles are liposomes.

The supramolecular cell carrier provided in this application can be used to deliver liposomes or cells. Drugs can be loaded in liposomes, that is, as nano-medicines, they are pulled by cells for targeted delivery, and the release mechanism is mainly related to the properties of liposomes themselves. Drug-loaded liposomes can be separated from the supramolecular cell carrier in the following ways. One is due to the fluidity of the cell membrane, and the other is that the drug-loaded liposomes are released by the carrier cells are directly phagocytized and digested by target cells, resulting in the release of intracellular drugs.

The drug-carrying system provided in this application can be used to load anti-inflammatory drugs, antibiotics, targeted cancers, and therapeutic agents for nervous system diseases.

The anti-inflammatory drug such as quercetin can be loaded in liposomes. After conjugation with supramolecular macrophage, it can be delivered to the pneumonia site to treat acute pneumonia.

The drug-loading system can be used to load doxorubicin.

The application has the following beneficial effects:

The application provides a supramolecular cell-based carrier, drug loading system and its preparation method. Host-guest interaction mediated supramolecular cell-based carriers can achieve targeted delivery based on the physiological function of transporting cell, and have high biocompatibility, high physiological barrier permeability, and high targeting efficiency. It does not require covalent modification on the cell surface, and has no effect on the physiological functions of modified cell. The preparation method of supramolecular cell-based carrier provided by this application has the advantages of simple and rapid preparation process, mild conditions, universal applicability, and bio-orthogonality. In addition, a drug loading system is also provided, which can realize drug loading for targeted therapy.

### BRIEF DESCRIPTION OF THE DRAWINGS

In order to more clearly illustrate the technical solutions of the embodiments of the present disclosure, the following will briefly introduce the accompanying drawings used in the embodiments. It is to be expressly understood that the following drawings only show some examples of the present disclosure, so they should not be intended as a definition of the limits of the invention. Those skilled in this art can also obtain other related drawings based on these drawings without creative work.

FIG. 1 is a fluorescence imaging diagram of the supramolecular cell-liposome conjugate in Example 1 of the present disclosure.

FIG. 2 is a scanning electron micrograph of the supramolecular cell-liposome conjugate in Example 1 of the present disclosure.

FIG. 3 is a fluorescent imaging diagram of the supramolecular cell-cell conjugate in Example 5 of the present disclosure.

FIG. 4 is a scanning electron microscope image of the supramolecular cell-cell conjugate in Example 5 of the present disclosure.

### DETAILED DESCRIPTION OF THE EMBODIMENTS

In order to make the purpose, technical solutions, and advantages of the embodiments of the present disclosure clearer, the technical solutions in the embodiments of the present disclosure will be clearly and completely described below. Those who do not indicate the specific conditions in the examples are carried out according to the conventional conditions or the conditions recommended by the manufacturer. The reagents or instruments without manufacturer information are all conventional products that could be purchased from the market.

The characteristics and performance of the present disclosure will be described in further detail below with examples.

### EXAMPLE 1

This example provides a supramolecular cell-based carrier and its preparation method. In this example, the first cell is macrophage, and both DSPE-PEG-β-CD and DSPE-PEG-ADA are purchased from Xi'an ruixi Biological Technology Co., Ltd., DMEM medium was purchased from Thermo Fisher Scientific (China) Co., Ltd., and doxorubicin was purchased from Shanghai Aladdin Biochemical Technology Co., Ltd.

Macrophages were incubated in a blank DMEM medium containing 10 μM of DSPE-PEG-β-CD at 37° C. for 2 hours.

After incubation, excess DSPE-PEG-β-CD was washed away, and 10 μM of DSPE-PEG-ADA-modified doxorubicin-loaded liposomes were added to further incubate for 2 minutes.

After washing off unbound liposomes, supramolecular cell-liposome conjugates were prepared.

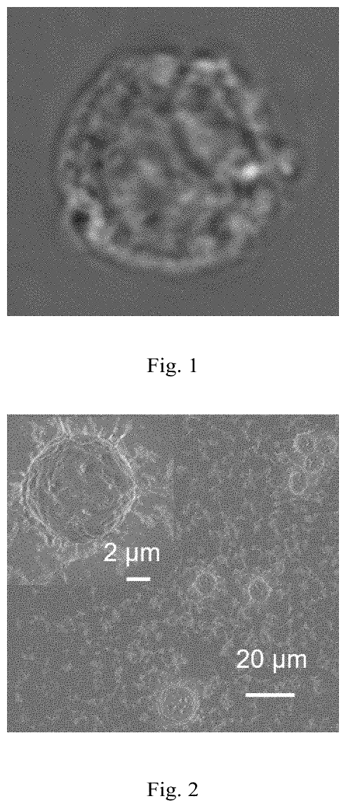

Since doxorubicin has red fluorescence, the prepared supramolecular cell-liposome conjugates were tracked by fluorescence imaging and scanning electron microscope imaging. The fluorescence imaging of the supramolecular cell-liposome conjugate is shown in FIG. 1, and the scanning electron microscope image of the supramolecular cell-liposome conjugate is shown in FIG. 2.

### EXAMPLE 2

This example provides a supramolecular cell-based carrier and its preparation method. In this example, DSPE-PEG-β-CD and DMEM medium were purchased from the same source in Example 1. In this example, the first cell is macrophage, and the macrophages were incubated in a blank DMEM medium containing 50 μM of DSPE-PEG-β-CD at 37° C. for 1 hour.

After washing off excess DSPE-PEG-β-CD, 50 μM of DSPE-PEG-ADA modified liposomes were added and incubated for 2 minutes. Finally, after removing unbound liposomes, supramolecular cell-liposome conjugates were obtained.

### EXAMPLE 3

This example provides a supramolecular cell-based carrier and its preparation method. DMPE-PEG-CB[7] (CB[7] is cucurbit[7]uril) and DMPE-PEG-ADA were prepared in laboratory. In this example, neutrophils were incubated in a blank DMEM medium containing 100 μM of DMPE-PEG-CB[7] at 37° C. for 2 hours.

After washing off excess DMPE-PEG-CB[7], 100 μM of DMPE-PEG-ADA modified liposomes were added and incubated for 1 minute. Finally, after removing unbound liposomes, supramolecular cell-liposome conjugates were obtained.

### EXAMPLE 4

This example provides a supramolecular cell-based carrier and its preparation method. DPPE-PEG-CB[7] and DPPE-PEG-ADA were prepared in laboratory. In this example, hematopoietic stem cells were incubated in a blank DMEM medium containing 40 μM of DPPE-PEG-CB[7] at 37° C. for 1.5 hours.

Then excess DPPE-PEG-CB[7] was washed off, and 80 μM of DPPE-PEG-ADA modified liposomes were added to continue incubation for 5 minutes. After washing off unbound liposomes, supramolecular cell-liposome conjugates were obtained.

### EXAMPLE 5

This example provides a supramolecular cell-based carrier and its preparation method. Both DiD and DiO were purchased from Shanghai Beyotime Biotechnology Co., Ltd. In this example, macrophages were incubated in a blank DMEM medium containing 10 μM of DSPE-PEG-β-CD at 37° C. for 2 hours. After washing off excess DSPE-PEG-β-CD, 10 μM of DSPE-PEG-ADA modified human hepatocytes were added and further incubated for 2 minutes. Then unbounded hepatocytes were washed off to obtain supramolecular cell-cell conjugates.

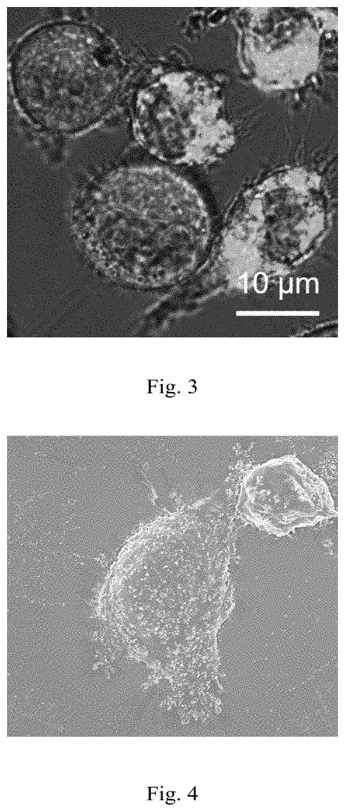

The obtained supramolecular cell-cell conjugates were studied by fluorescence imaging and scanning electron microscope imaging. The fluorescence imaging is shown in FIG. 3, and the scanning electron microscope imaging is shown in FIG. 4. Macrophages were stained with DiD (red) and human hepatocytes were stained with DiO (green).

### EXAMPLE 6

This example provides a supramolecular cell-based carrier and its preparation method. Fc was purchased from Shanghai Aladdin Biochemical Technology Co., Ltd., and DSPE-PEG-Fc was synthesized in laboratory. In this example, macrophages were incubated in a blank DMEM medium containing 10 μM of DSPE-PEG-β-CD at 37° C. for 2 hours. After incubation, excess DSPE-PEG-β-CD was washed off, and 10 μM of DSPE-PEG-Fc (Fc was ferrocene) modified human hepatocytes were added to incubate for 2 minutes. Then unbound cells were washed off to obtain supramolecular cell-cell conjugates.

### EXAMPLE 7

This example provides a supramolecular cell-based carrier and its preparation method. DMPE-PEG-P5 (P5 is Pillar[5]arene) was synthesized in laboratory. In this example, neutrophils were incubated at 37° C. for 2 hours in a blank DMEM medium containing 60 μM of DMPE-PEG-P5. After incubation, excess DMPE-PEG-P5 was washed off, and then 30 μM of DSPE-PEG-Fc (Fc is ferrocene) modified red blood cells were added and continued to incubate for another 2 minutes. After removing unbound cells, cell-cell conjugates were obtained.

### EXAMPLE 8

This example provides a supramolecular cell-based carrier and its preparation method. Hematopoietic stem cells are derived from the American Type Culture Collection (ATCC), and both DPPE-PEG-β-CD and DPPE-PEG-ADA were prepared in laboratory. In this example, hematopoietic stem cells were incubated in a blank DMEM medium containing 100 μM of DPPE-PEG-β-CD at 37° C. for 2 hours. After washing off excess DPPE-PEG-β-CD, 150 of μM DPPE-PEG-ADA-modified red blood cells were added and further incubated with resulting hematopoietic stem cells for 2 minutes. After washing off unbound cells, cell-cell conjugates were obtained.

### COMPARATIVE EXAMPLE

Embryonic stem cells were incubated in a blank DMEM medium containing 10 μM of DPPE-PEG-CB[7] at 37° C. for 5 minutes. After washing away excess DPPE-PEG-CB[7], 10 μM of DPPE-PEG-ADA modified doxorubicin-loaded liposomes were added to further incubate for 5 minutes. After washing off the unbound liposomes, fluorescence imaging was performed, and no red fluorescence was found on the embryonic stem cell membrane.

In summary, the supramolecular cell-based carrier of the embodiment of present disclosure is constructed through host-guest interaction, and is a new generation of cell-based formulations, which can achieve targeted delivery based on the inherent physiological function of transporting cell; The preparation method of supramolecular cell-based carrier is that macrocyclic host molecule coupled with membrane-embedding material anchored on the first cell surface via membrane insertion, and then mixing with the nanoparticles modified with guest molecule or the second cells modified with guest molecule. The preparation process is cell friendly, facile, universal and bioorthogonal, which does not require any covalent modification on the cell surface and has no effect on the physiological function of the modified cell.

The above descriptions are only preferred examples of the present disclosure and are not intended to limit the present disclosure. For those skilled in the art, there may be various modifications and changes in the present disclosure. Any modifications, equivalent replacements, improvements, etc. made within the spirit and principles of this disclosure shall be included within the protection scope of this disclosure.

What is claimed is:

1. A supramolecular cell-based carrier, comprising: a first part and a second part conjugated to each other through host-guest interaction, wherein the first part is a first cell modified by a macrocyclic host molecule or guest molecule, and the second part is a nanoparticle modified by the guest molecule or macrocyclic host molecule, or the second cell modified by the guest molecule or macrocyclic host molecule, the macrocyclic host molecule or guest molecule in the first part is embedded in the cell membrane of the first cell by coupling a membrane-embedding material, the macrocyclic host molecule or guest molecule is in correspondence with the guest molecule or macrocyclic host molecule.

2. The supramolecular cell-based carrier of claim 1, wherein the macrocyclic main molecule is cyclodextrin, cucurbituril, calixarene, pillararene or crown ether.

3. The supramolecular cell-based carrier of claim 1, wherein the first cell is selected from macrophage, neutrophil, red blood cell, stem cell, lymphocyte, dendritic cell, platelet and fat cell.

4. The supramolecular cell-based carrier of claim 1, wherein the molar ratio of the macrocyclic host molecule to the guest molecule is 1-10:1-10, the guest molecule is adamantane or ferrocene.

5. The supramolecular cell-based carrier of claim 1, wherein the nanoparticles are at least one of liposomes, micelles, nanogels, inorganic nanoparticles and nanocapsules, the second cells are liver cells, stem cells, lymphocytes, dendritic cells, platelets, fat cells or red blood cells.

6. The supramolecular cell-based carrier of claim 5, wherein the membrane-embedding material is PEG-DMPE, PEG-DPPE, PEG-DSPE or PEG-CHOL.

7. A method for preparing the supramolecular cell-based carrier of claim 1, comprising:
(a) co-incubating the macrocyclic host molecule or guest molecule coupled with the membrane-embedding material with the first cell to obtain the first part; and
(b) then mixing the nanoparticles modified with the guest molecules or macrocyclic host molecule, or the second cells modified with the guest molecules or macrocyclic host molecule with the first part.

8. A method for preparing the supramolecular cell-based carrier of claim 2, comprising:
(a) co-incubating the macrocyclic host molecule or guest molecule coupled with the membrane-embedding material with the first cell to obtain the first part; and
(b) then mixing the nanoparticles modified with the guest molecules or macrocyclic host molecule, or the second cells modified with the guest molecules or macrocyclic host molecule with the first part.

9. A method for preparing the supramolecular cell-based carrier of claim 3, comprising:
(a) co-incubating the macrocyclic host molecule or guest molecule coupled with the membrane-embedding material with the first cell to obtain the first part; and
(b) then mixing the nanoparticles modified with the guest molecules or macrocyclic host molecule, or the second cells modified with the guest molecules or macrocyclic host molecule with the first part.

10. A method for preparing the supramolecular cell-based carrier of claim 4, comprising:
(a) co-incubating the macrocyclic host molecule or guest molecule coupled with the membrane-embedding material with the first cell to obtain the first part; and
(b) then mixing the nanoparticles modified with the guest molecules or macrocyclic host molecule, or the second cells modified with the guest molecules or macrocyclic host molecule with the first part.

11. A method for preparing the supramolecular cell-based carrier of claim 5, comprising:
(a) co-incubating the macrocyclic host molecule or guest molecule coupled with the membrane-embedding material with the first cell to obtain the first part; and
(b) then mixing the nanoparticles modified with the guest molecules or macrocyclic host molecule, or the second cells modified with the guest molecules or macrocyclic host molecule with the first part.

12. A method for preparing the supramolecular cell-based carrier of claim 6, comprising:
(a) co-incubating the macrocyclic host molecule or guest molecule coupled with the membrane-embedding material with the first cell to obtain the first part; and
(b) then mixing the nanoparticles modified with the guest molecules or macrocyclic host molecule, or the second cells modified with the guest molecules or macrocyclic host molecule with the first part.

13. The preparation method of claim 7, wherein the preparation method further comprises covalently conjugating the macrocyclic molecule or guest molecule to the membrane-embedding material, and then embedding the macrocyclic molecule or guest molecule in the cell membrane of the first cell through membrane-embedding, the macrocyclic host molecule or guest molecule is covalently linked to the PEG of membrane-embedding material, the macrocyclic host molecule or guest molecule covalently conjugated with membrane-embedding material is co-incubated with the first cell for more than 30 minutes, the concentration of the macrocyclic host molecule or guest molecule covalently conjugated with membrane-embedding material is 1 μM-1 mM.

14. The preparation method of claim 7, wherein the time for mixing and incubating the nanoparticles modified with guest molecules or macrocyclic host molecule, or the second cells modified with guest molecules or macrocyclic host molecule with the first part is ≥10 seconds.

15. A drug-carrying system, comprising the supramolecular cell-based carrier of claim 1 and a drug, wherein the drug is loaded in the nanoparticle or in the second cell; the nanoparticle is a liposome.

16. A drug-carrying system, comprising the supramolecular cell-based carrier of claim 2 and a drug, wherein the drug is loaded in the nanoparticle or in the second cell; the nanoparticle is a liposome.

17. A drug-carrying system, comprising the supramolecular cell-based carrier of claim 3 and a drug, wherein the drug is loaded in the nanoparticle or in the second cell; the nanoparticle is a liposome.

18. A drug-carrying system, comprising the supramolecular cell-based carrier of claim 4 and a drug, wherein the drug is loaded in the nanoparticle or in the second cell; the nanoparticle is a liposome.

19. A drug-carrying system, comprising the supramolecular cell-based carrier of claim 5 and a drug, wherein the drug is loaded in the nanoparticle or in the second cell; the nanoparticle is a liposome.

20. A drug-carrying system, comprising the supramolecular cell-based carrier of claim 6 and a drug, wherein the drug is loaded in the nanoparticle or in the second cell; the nanoparticle is a liposome.
